(19) United States
(12) Patent Application Publication
(10) Pub. No.: US 2021/0082581 A1
(43) Pub. Date: Mar. 18, 2021

(54) DETERMINING NOVELTY OF A CLINICAL TRIAL AGAINST AN EXISTING TRIAL CORPUS

(71) Applicant: INTERNATIONAL BUSINESS MACHINES CORPORATION, Armonk, NY (US)

(72) Inventors: Kyle G. Christianson, Rochester, MN (US); Eric L. Erpenbach, Oronoco, MN (US); Tyra Alexa Mccoy, Ellenwood, GA (US); Katherine A. Kairis, Pittsburgh, PA (US)

(21) Appl. No.: 16/572,991

(22) Filed: Sep. 17, 2019

(51) Int. Cl. G16H 50/70; G16H 10/20; G16H 15/00

(52) U.S. Cl. CPC G16H 50/70; G16H 15/00; G16H 10/20

(57) ABSTRACT

A method, computer system, and computer program product for measuring the similarity of clinical trials are provided. The embodiment may include receiving user-entered clinical trial data. The embodiment may also include extracting concepts and values from each section of the received clinical trial data using natural language processing. The embodiment may further include searching a corpus of existing trials with concepts similar to the extracted concepts and values. The embodiment may also include computing an edit-distance for each section of the existing trial. The embodiment may further include calculating an overall similarity score based on a weighted distance of each section of the existing trial. The embodiment may also include displaying the overall similarity score to a user on a graphical user interface.

### BACKGROUND

The present invention relates, generally, to the field of computing, and more particularly to document similarity analysis.

Document similarity analysis generally involves extracting a document vector to represent the documents as a whole using a statistical approach. The vector is made from the statistically most important words contained in the document. Vocabularies contained in a document may also be analyzed to obtain a document vector when a specific topic is the main factor in comparing two different documents. The importance of vocabularies or terms is often weighted in accordance with its frequencies in data set as a whole. After document vectors are extracted, then the information is stored as metadata in a database such that similarity analysis may perform comparing the vectors of different documents. Cosine similarity is a commonly used similarity measure for real-valued vectors in information retrieval to score the similarity of different documents. Today, in machine learning, common kernel functions such as the radial basis function (RBF) kernel can be commonly used in support vector machine classification.

### SUMMARY

According to one embodiment, a method, computer system, and computer program product for measuring the similarity of clinical trials are provided. The embodiment may include receiving user-entered clinical trial data. The embodiment may also include extracting concepts and values from each section of the received clinical trial data using natural language processing. The embodiment may further include searching a corpus of existing trials with concepts similar to the extracted concepts and values. The embodiment may also include computing an edit-distance for each section of the existing trial. The embodiment may further include calculating an overall similarity score based on a weighted distance of each section of the existing trial. The embodiment may also include displaying the overall similarity score to a user on a graphical user interface.

### BRIEF DESCRIPTION OF THE SEVERAL VIEWS OF THE DRAWINGS

These and other objects, features, and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment;

FIG. 2 is an operational flowchart illustrating a clinical trial similarity calculation process according to at least one embodiment;

FIG. 3 is an operational flowchart illustrating a matching or edit distance algorithm processed around each sections of clinical trials according to at least one embodiment;

FIG. 4 is a block diagram of internal and external components of computers and servers depicted in FIG. 1 according to at least one embodiment;

FIG. 5 depicts a cloud computing environment according to an embodiment of the present invention; and

FIG. 6 depicts abstraction model layers according to an embodiment of the present invention.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Embodiments of the present invention relate to the field of computing, and more particularly to document similarity analysis. The following described exemplary embodiments provide a system, method, and program product to determine the similarity of clinical trials data by breaking down clinical trials into key components using NLP (Natural Language Processing). A weight is applied to each concept based on its location and context of the concept in a document and stored in an indexed corpus. With this data, a similarity calculation may be determined for each section of trails by taking into account direct matching, the difference in the distance length from a common parent node, and the difference in the location or importance of the concept within the document. Therefore, the present embodiment has the capacity to improve the technical field of clinical trial document similarity analysis systems by identifying the similarities or differences between various aspects of clinical trials with higher accuracy and efficiency.

As previously described, document similarity analysis generally involves extracting a document vector to represent the documents as a whole using a statistical approach. The vector is made from the statistically most important words contained in the document. Vocabularies contained in a document may also be analyzed to obtain a document vector when a specific topic is the main factor in comparing two different documents. The importance of vocabularies or terms is often weighted in accordance with its frequencies in data set as a whole. After document vectors are extracted, then the information is stored as metadata in a database such that similarity analysis may perform comparing the vectors of different documents. Cosine similarity is a commonly used similarity measure for real-valued vectors in information retrieval to score the similarity of different documents. Today, in machine learning, common kernel functions such as the radial basis function (RBF) kernel can be commonly used in support vector machine classification.

Vital medications and treatments are often developed through clinical trial research, and the time and cost associated with clinical trial research are burdening. A single trial takes years to complete and may cost millions of dollars to develop. Often, clinical trial designers spend hundreds of hours manually researching past trials to analyze historical information, in an effort to find similarities to assist in understanding what has been attempted in the past in order to understand the risk or what is unique with respect to the current clinical study. Also, this critical research stage can be time-consuming and error-prone. Each clinical trial document can be well over 100 pages. A review may require clinical designers to spend a significant amount of time reading through the protocol and comparing the different sections and recognizing the different concepts. Details may be buried in different sections and even though the same concepts could be found, their meanings may vary depending on the context. As such, it may be advantageous to, among other things, implement a system capable of estimating the novelty of a proposed clinical trial by comparing its key sections to the sections of existing trials in a corpus. This similarity analysis involves employing NLP techniques, along with a distance algorithm that calculates the similarity between two trials by comparing their concepts' hierarchical relationships, locations within the trials, and importance within the trials. As a result, the current invention may save time for clinical trial designers by identifying the similarities or differences between various aspects of clinical trials with higher accuracy and efficiency.

According to one embodiment, the present invention may extract certain concepts from each section of a provided clinical trial using NLP. In at least one other embodiment, the present invention may also search the corpus for existing trials whose concepts either match or have immediate relationships to the concepts extracted from the provided trial. The present invention may further determine the most similar existing trials and generate a summary of the provided trial's overall similarity against the existing trials in the corpus.

The present invention may be a system, a method, and/or a computer program product at any possible technical detail level of integration. The computer program product may include the computer readable storage medium (or media) having computer readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer-readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or another device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method, and program product for measuring the similarity of documents in a corpus based on the computation of the distance between two concept values using a distance function.

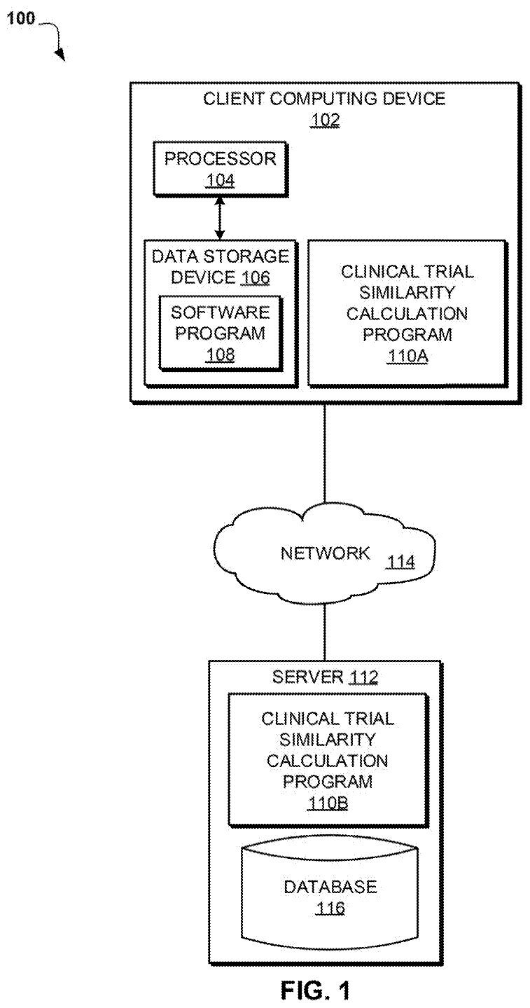

Referring to FIG. 1, an exemplary networked computer environment 100 is depicted according to at least one embodiment. The networked computer environment 100 may include client computing device 102 and a server 112 interconnected via a communication network 114. According to at least one implementation, the networked computer environment 100 may include a plurality of client computing devices 102 and servers 112 of which only one of each is shown for illustrative brevity.

The communication network 114 may include various types of communication networks, such as a wide area network (WAN), local area network (LAN), a telecommunication network, a wireless network, a public switched network and/or a satellite network. The communication network 114 may include connections, such as wire, wireless communication links, or fiber optic cables. It may be appreciated that FIG. 1 provides only an illustration of one implementation and does not imply any limitations with regard to the environments in which different embodiments may be implemented. Many modifications to the depicted environments may be made based on design and implementation requirements.

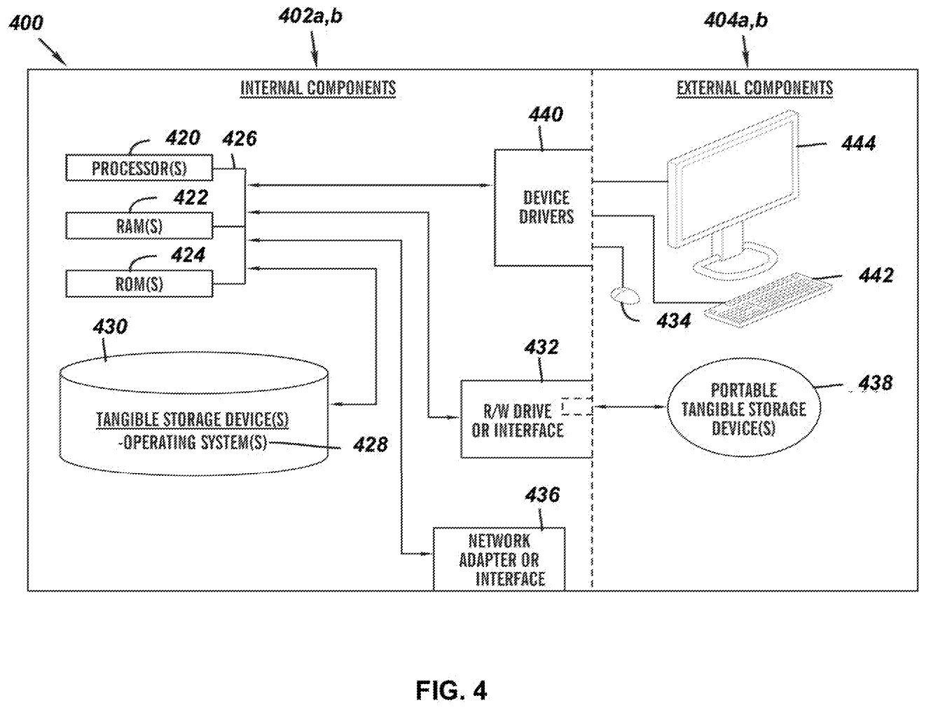

Client computing device 102 may include a processor 104 and a data storage device 106 that is enabled to host and run a software program 108 and a clinical trial similarity calculation program 110A and communicate with the server 112 via the communication network 114, in accordance with one embodiment of the invention. Client computing device 102 may be, for example, a mobile device, a telephone, a personal digital assistant, a netbook, a laptop computer, a tablet computer, a desktop computer, or any type of computing device capable of running a program and accessing a network. As will be discussed with reference to FIG. 4, the client computing device 102 may include internal components 402a and external components 404a, respectively.

The server computer 112 may be a laptop computer, netbook computer, personal computer (PC), a desktop computer, or any programmable electronic device or any network of programmable electronic devices capable of hosting and running a clinical trial similarity calculation program 110B and a database 116 and communicating with the client computing device 102 via the communication network 114, in accordance with embodiments of the invention. As will be discussed with reference to FIG. 4, the server computer 112 may include internal components 402b and external components 404b, respectively. The server 112 may also operate in a cloud computing service model, such as Software as a Service (SaaS), Platform as a Service (PaaS), or Infrastructure as a Service (IaaS). The server 112 may also be located in a cloud computing deployment model, such as a private cloud, community cloud, public cloud, or hybrid cloud.

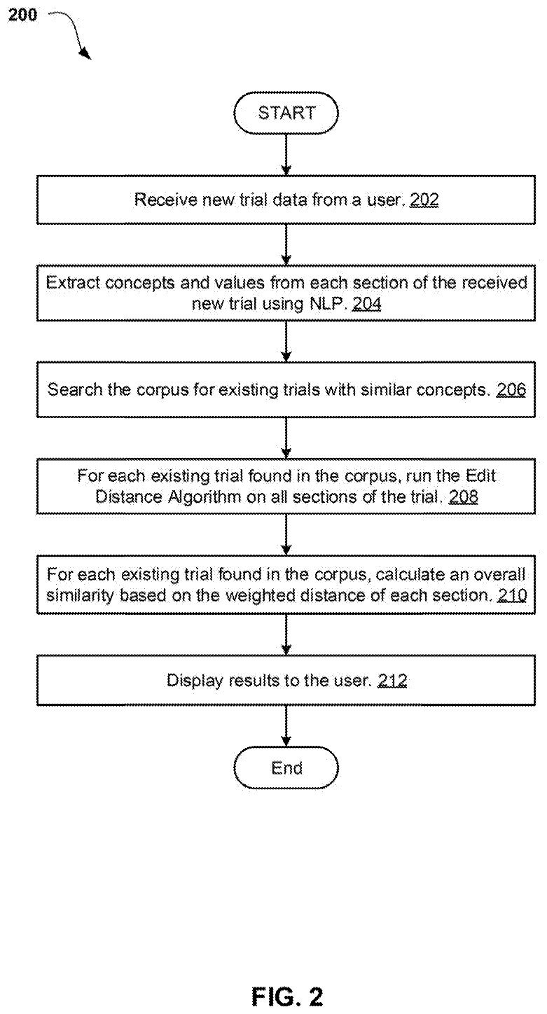

According to the present embodiment, the clinical trial similarity calculation program 110A, 110B may be a program capable of calculating an overall similarity of extracted concepts based on the weighted distance of each section of a clinical trial. The clinical trial similarity calculation process is explained in further detail below with respect to FIG. 2.

Referring to FIG. 2, an operational flowchart illustrating a clinical trial similarity calculation process 200 is depicted according to at least one embodiment. At 202, the clinical trial similarity calculation program 110A, 110B receives new trial data from a user. According to one embodiment, a user may upload one or more documents containing trial data. For example, a user may upload such a document in any format such as word document, pdf, jpeg, video, etc. In at least one other embodiment, a corpus may be set up with existing clinical trials, such as trials from an available database. For example, if a user wants to determine similarities between clinical trials involving a drug that alleviates high blood pressure, a user may manually upload related documents or the clinical trial similarity calculation program 110A, 110B may search databases for documents that are relevant to the new clinical trial provided by the user.

At 204, the clinical trial similarity calculation program 110A, 110B extracts concepts and values from each section of the received new trial using NLP. For example, if the sections of the received new trial contain the concepts “time frame” and “blood pressure,” the clinical trial similarity calculation program 110A, 110B may use NLP to extract these two concepts from the new trial, in addition to any other concepts that occur in all sections of this trial. In at least one other embodiment, the clinical trial similarity calculation program 110A, 110B may index each concept extracted from new documents or the existing corpus for easy searching in later steps.

At 206, the clinical trial similarity calculation program 110A, 110B searches the corpus for existing trials with similar concepts. According to one embodiment, the clinical trial similarity calculation program 110A, 110B may extract particular concepts from each section of a new trial and then search the corpus for existing trials whose concepts are similar to the concepts that appear in the new trial. Two concepts may be considered similar if they are either the same concept, or if they have a parent-child relationship in a concept ontology. For example, if the clinical trial similarity calculation program 110A, 110B extracts the “diabetes” concept from a received trial, it may then search the existing corpus for trial documents that contain concepts related to “diabetes”. Existing trials in the corpus would match not only if they contain the “diabetes” concept, but also if they contain any parent or child concept of “diabetes,” such as “type 1 diabetes”, “type 2 diabetes”, and “prediabetes.”.

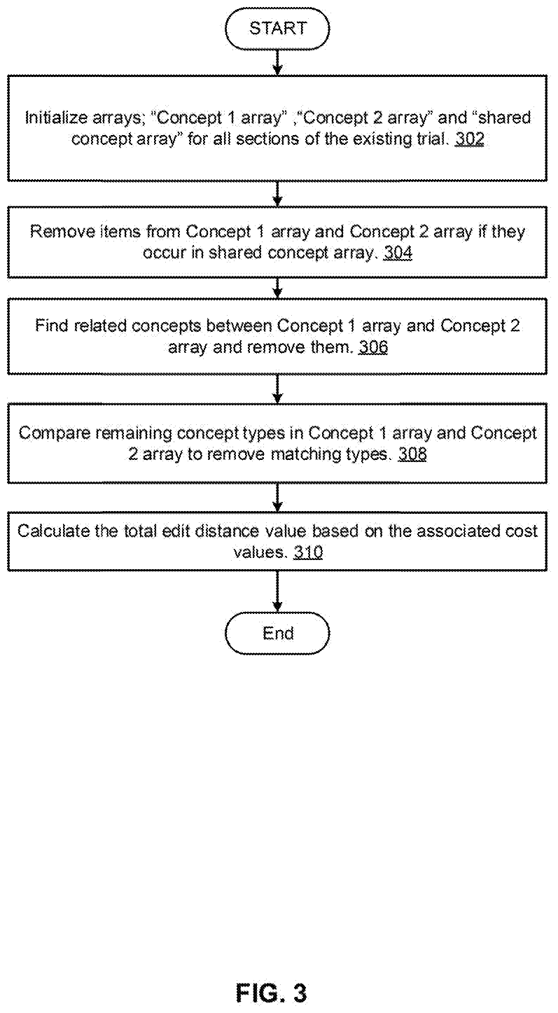

At 208, the clinical trial similarity calculation program 110A, 110B runs an edit distance algorithm to compare each section of the new trial with the sections of the existing trials found at 206. According to one embodiment, the clinical trial similarity calculation program 110A, 110B may compare the concepts that occur in two different text snippets. The algorithm may compute the similarity between two trials as a version of edit distance; it may represent the two trials as two lists that contain the concepts in each trial, then calculate the cost of transforming the first trial's concept list into the second trial's concept list. Therefore, a lower edit distance may indicate a greater degree of similarity between the two trials. The edit distance algorithm is explained in further detail below with respect to FIG. 3.

At 210, the clinical trial similarity calculation program 110A, 110B calculates the overall similarities between the new trial provided by the user and each existing trial found at 206. The overall similarity score is computed by weighting the sections' similarity scores, which are calculated at 208, based on the sections' importance. According to one embodiment, the clinical trial similarity calculation program 110A, 110B may determine that, based on pre-configured criteria, certain trial sections have more importance than other sections. For example, the section that describes a trial's primary outcome will likely have more importance than the sections describing the trial's secondary and tertiary outcomes. As such, when assigning weights used in calculating the overall similarity of trials, the weight of the primary outcome section may be greater than the weights assigned to both the secondary and tertiary outcomes.

At 212, the clinical trial similarity calculation program 110A, 110B display results to a user. According to one embodiment, the clinical trial similarity calculation program 110A, 110B may generate a summary of the calculated overall similarity and provide a user with a summary report. In at least one other embodiment, the clinical trial similarity calculation program 110A, 110B may transmit the most similar existing trials to a user along with the summary of the overall trial's similarities against the entire corpus.

Referring to FIG. 3, an operational flowchart illustrating a matching or edit distance algorithm processed around each section of a clinical trial is depicted according to at least one embodiment. At 302, the clinical trial similarity calculation program 110A, 110B initializes arrays. More specifically, the clinical trial similarity calculation program 110A, 110B initializes, “concept 1 array” with concepts occurring in the first trial; “concept 2 array” with concepts occurring in the second trial; and “shared concepts array” which contains concepts occurring in both trials. According to one embodiment, an operational flowchart illustrating a matching or edit distance algorithm according to at least one embodiment may compute the edit distance between two trials by matching each concept in “concepts 1 array” with concepts in “concepts 2 array”. Concepts may be removed from their respective lists if they are matched in some way with concepts from the opposite list. In at least one other embodiment, the clinical trial similarity calculation program 110A, 110B may determine that a concept is considered to have a match in any of the flowing cases: the exact concept occurs in the opposite trial, a related concept occurs in the opposite trial, or a concept with the same semantic type occurs in the opposite trial. In many cases, a cost may be associated with each removal of matched concepts. Such cost may be added to the overall edit distance at the end of the algorithm. Additionally, the cost added to the overall edit distance may vary depending on how the concepts were matched—the cost of removing identical concepts from each list may be lower than the cost of removing two different concepts that only share semantic type. In at least one other embodiment, the clinical trial similarity calculation program 110A, 110B may initialize arrays with two lists of concepts 1 and concepts 2 extracted from a corpus. Concepts 1 may be a list of all of the concepts referred to in the first trial and duplicate concepts may be removed from the list. Concepts 2 may be a list of all of the concepts referred to in the second trail and duplicate concepts may be removed from the list as well. The clinical trial similarity calculation program 110A, 110B may create a new list for shared concepts that contain the concepts found in both Concepts 1 and Concepts 2.

At 304, the clinical trial similarity calculation program 110A, 110B removes items from Concept 1 array and Concept 2 array if they occur in the shared concept array and accumulates the costs associated with these removals. According to one embodiment, the clinical trial similarity calculation program 110A, 110B may determine that a small cost is added to the edit distance if a concept is negated in one trial, but not in the other trial. A small cost may be added to the overall edit distance if the values associated with the concept differ between two trials. The clinical trial similarity calculation program 110A, 110B may accumulate such costs calculated for each item that may be removed from one array. The accumulated cost may be used to calculate the overall similarity in the later steps below. In at least one other embodiment, a cost may not be added to the edit distance if two concepts match.

At 306, the clinical trial similarity calculation program 110A, 110B removes concepts from their respective arrays if a related concept occurs in the opposite array. This step utilizes an ontology to find concepts that are related to the remaining concepts in Concept 1 array and Concept 2 array. In at least one other embodiment, related concepts may only include parents and children up to 3 positions away from the concept in question. In at least one other embodiment, the clinical trial similarity calculation program 110A, 110B may include other relations, such as siblings of a concept, and adjust the maximum distance between the given concept and a relative. The clinical trial similarity calculation program 110A, 110B may remove items from Concept 1 if there is a related concept in Concept 2 or vice versa. In one embodiment, the clinical trial similarity calculation program 110A, 110B may set a cost of removal of a related concept is half the distance between a concept in one list and a related concept in the opposite list. In yet other embodiment, a cost may depend on the type of relationship (e.g. different costs for parents vs. children or siblings). At the end of step 306, the concepts remaining in Concept 1 and Concept 2 occur only in their respective trials and may not have a related concept in the opposite trial. The clinical trial similarity calculation program 110A, 110B may then create two new lists for Types 1 and Types 2. Types 1 may be a list of the semantic types of the remaining concepts in Concept 1 and Types 2 is a list of the semantic types of the remaining concepts in Concept 2.

At 308, the clinical trial similarity calculation program 110A, 110B compares the semantic types of the remaining concepts in Concept 1 array and Concept 2 array and removes matching types with accumulated cost values. According to one embodiment, the clinical trial similarity calculation program 110A, 110B may compare the semantic types of the concepts that have not been matched—at this step, each remaining concept in Concept 1 and Concept 2 does not occur in the opposite trial and does not have a related concept in the opposite trial. In at least one other embodiment, the clinical trial similarity calculation program 110A, 110B may check for shared concept “types” between Types 1 and Types 2 and when there is a shared type, the clinical trial similarity calculation program 110A, 110B may remove one instance of such type from each list. With respect to an edit distance cost, the cost to the edit distance may be directly related to the weight of the semantic type. The weights of the semantic types may be predetermined and depend on how important each semantic type is to clinical trials.

At 310, the clinical trial similarity calculation program 110A, 110B calculates the total edit distance value based on the associated cost values. According to one embodiment, the clinical trial similarity calculation program 110A, 110B may compute cost values for each section combine the cost values to determine the overall strength of a match. The end result of this step may indicate the total cost of transforming the first trial's list of concepts into the second trial's list of concepts, and thus, a low edit distance may indicate a greater degree of similarity between the two trials.

It may be appreciated that FIGS. 2-3 provide only an illustration of one implementation and do not imply any limitations with regard to how different embodiments may be implemented. Many modifications to the depicted environments may be made based on design and implementation requirements. For example, in at least one embodiment, the clinical trial similarity calculation program 110A, 110B may compute and update an edit distance value as additional trials are added to a corpus.

FIG. 4 is a block diagram of internal and external components of the client computing device 102 and the server 112 depicted in FIG. 1 in accordance with an embodiment of the present invention. It should be appreciated that FIG. 4 provides only an illustration of one implementation and does not imply any limitations with regard to the environments in which different embodiments may be implemented. Many modifications to the depicted environments may be made based on design and implementation requirements.

The data processing system 402, 404 is representative of any electronic device capable of executing machine-readable program instructions. The data processing system 402, 404 may be representative of a smartphone, a computer system, PDA, or other electronic devices. Examples of computing systems, environments, and/or configurations that may represented by the data processing system 402, 404 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

The client computing device 102 and the server 112 may include respective sets of internal components 402 a,b and external components 404 a,b illustrated in FIG. 4. Each of the sets of internal components 402 include one or more processors 420, one or more computer-readable RAMs 422, and one or more computer-readable ROMs 424 on one or more buses 426, and one or more operating systems 428 and one or more computer-readable tangible storage devices 430. The one or more operating systems 428, the software program 108 and the clinical trial similarity calculation program 110A in the client computing device 102 and the clinical trial similarity calculation program 110B in the server 112 are stored on one or more of the respective computer-readable tangible storage devices 430 for execution by one or more of the respective processors 420 via one or more of the respective RAMs 422 (which typically include cache memory). In the embodiment illustrated in FIG. 4, each of the computer-readable tangible storage devices 430 is a magnetic disk storage device of an internal hard drive. Alternatively, each of the computer-readable tangible storage devices 430 is a semiconductor storage device such as ROM 424, EPROM, flash memory or any other computer-readable tangible storage device that can store a computer program and digital information.

Each set of internal components 402 a,b also includes an R/W drive or interface 432 to read from and write to one or more portable computer-readable tangible storage devices 438 such as a CD-ROM, DVD, memory stick, magnetic tape, magnetic disk, optical disk or semiconductor storage device. A software program, such as the clinical trial similarity calculation program 110A, 110B can be stored on one or more of the respective portable computer-readable tangible storage devices 438, read via the respective R/W drive or interface 432 and loaded into the respective hard drive 430.

Each set of internal components 402 a,b also includes network adapters or interfaces 436 such as a TCP/IP adapter cards, wireless Wi-Fi interface cards, or 3G or 4G wireless interface cards or other wired or wireless communication links. The software program 108 and the clinical trial similarity calculation program 110A in the client computing device 102 and the clinical trial similarity calculation program 110B in the server 112 can be downloaded to the client computing device 102 and the server 112 from an external computer via a network (for example, the Internet, a local area network or other, wide area network) and respective network adapters or interfaces 436. From the network adapters or interfaces 436, the software program 108 and the clinical trial similarity calculation program 110A in the client computing device 102 and the clinical trial similarity calculation program 110B in the server 112 are loaded into the respective hard drive 430. The network may comprise copper wires, optical fibers, wireless transmission, routers, firewalls, switches, gateway computers and/or edge servers.

Each of the sets of external components 404 a,b can include a computer display monitor 444, a keyboard 442, and a computer mouse 434. External components 404 a,b can also include touch screens, virtual keyboards, touch pads, pointing devices, and other human interface devices. Each of the sets of internal components 402 a,b also includes device drivers 440 to interface to computer display monitor 444, keyboard 442, and computer mouse 434. The device drivers 440, R/W drive or interface 432, and network adapter or interface 436 comprise hardware and software (stored in storage device 430 and/or ROM 424).

It is understood in advance that although this disclosure includes a detailed description on cloud computing, implementation of the teachings recited herein is not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is a service oriented with a focus on statelessness, low coupling, modularity, and semantic interoperability. At the heart of cloud computing is an infrastructure comprising a network of interconnected nodes.

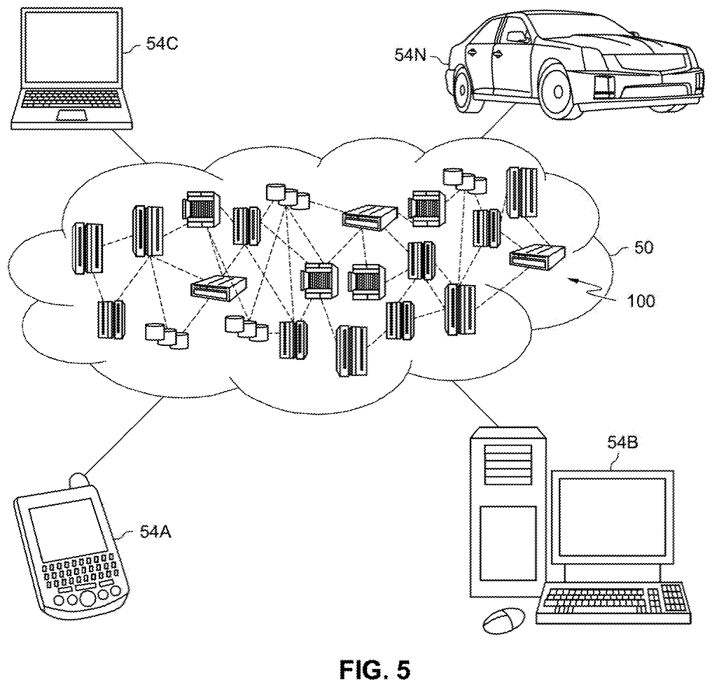

Referring now to FIG. 5, illustrative cloud computing environment 50 is depicted. As shown, cloud computing environment 50 comprises one or more cloud computing nodes 100 with which local computing devices used by cloud consumers, such as, for example, personal digital assistant (PDA) or cellular telephone 54A, desktop computer 54B, laptop computer 54C, and/or automobile computer system 54N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 100 and cloud computing environment 50 can communicate with any type of computerized device over any type of network and/or network addressable connection (e.g., using a web browser).

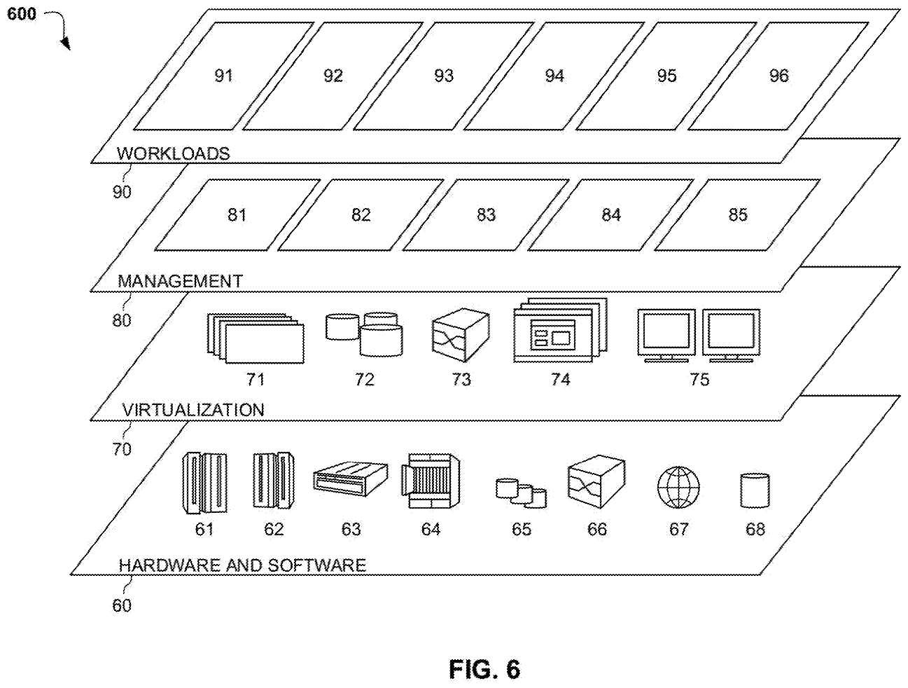

Referring now to FIG. 6, a set of functional abstraction layers 600 provided by cloud computing environment 50 is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and clinical trial similarity calculation 96. Clinical trial similarity calculation 96 may relate to determining similarities or differences of various aspect of a clinical trial based on the calculated distance length from a common parent note.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method for measuring the similarity of clinical trials, the method comprising:
receiving user-entered clinical trial data;
extracting concepts and values from each section of the received clinical trial data using natural language processing;
searching a corpus of existing trials with concepts similar to the extracted concepts and values;
computing an edit distance for each section of the existing trial;
calculating an overall similarity score based on a weighted distance of each section of the existing trial; and
displaying the overall similarity score to a user on a graphical user interface.

2. The method of claim 1, further comprising:
initializing arrays, wherein arrays consist of a first array for a concept of a first trial, a second array for the second concept of a second trial, and a shared concept array for shared concepts between the first and second trial;
removing items from the first array for the concept of the first trial and the second array for the second concept of the second trial;
calculating an edit cost for each removed item; and
accumulating the calculated edit cost.

3. The method of claim 1, further comprising:
using ontology to find related concepts between a first array for the concept of the first trial and the second array for the second concept of the second trial;
removing the shared concepts from the first array for the concept of the first trial and the second array for the second concept of the second trial;
calculating an edit cost for each removed item; and
accumulating the calculated edit cost.

4. The method of claim 1, further comprising:
comparing concepts remaining in a first array for the concept of the first trial and the second array for the second concept of the second trial, wherein the remaining concepts are concepts other than the shared concepts and matched concepts;
removing the remaining concepts from the first array for the concept of the first trial and the second array for the second concept of the second trial;
calculating an edit cost for each removed item; and
accumulating the calculated edit cost.

5. The method of claim 1, further comprising:
generating a summary of the calculated overall similarity and provide the user with a summary report.

6. The method of claim 1, further comprising:
determining that a lower edit distance value indicates a greater degree of similarity between the first trial and the second trial.

7. The method of claim 1, wherein immediate parents or children of the extracted concepts are searched together when the corpus of the existing trial is searched.

8. A computer system for measuring the similarity of clinical trials, the computer system comprising:
one or more processors, one or more computer-readable memories, one or more computer-readable tangible storage media, and program instructions stored on at least one of the one or more tangible storage media for execution by at least one of the one or more processors via at least one of the one or more memories, wherein the computer system is capable of performing a method comprising:
receiving user-entered clinical trial data;
extracting concepts and values from each section of the received clinical trial data using natural language processing;
searching a corpus of existing trials with concepts similar to the extracted concepts and values;
computing an edit distance for each section of the existing trial;
calculating an overall similarity score based on a weighted distance of each section of the existing trial; and
displaying the overall similarity score to a user on a graphical user interface.

9. The computer system of claim 8, further comprising:
initializing arrays, wherein arrays consist of a first array for a concept of a first trial, a second array for the second concept of a second trial, and a shared concept array for shared concepts between the first and second trial;
removing items from the first array for the concept of the first trial and the second array for the second concept of the second trial;
calculating an edit cost for each removed item; and
accumulating the calculated edit cost.

10. The computer system of claim 8, further comprising:
using ontology to find related concepts between a first array for the concept of the first trial and the second array for the second concept of the second trial;
removing the shared concepts from the first array for the concept of the first trial and the second array for the second concept of the second trial;
calculating an edit cost for each removed item; and
accumulating the calculated edit cost.

11. The computer system of claim 8, further comprising:
comparing concepts remaining in a first array for the concept of the first trial and the second array for the second concept of the second trial, wherein the remaining concepts are concepts other than the shared concepts and matched concepts;
removing the remaining concepts from the first array for the concept of the first trial and the second array for the second concept of the second trial;
calculating an edit cost for each removed item; and
accumulating the calculated edit cost.

12. The computer system of claim 8, further comprising:
generating a summary of the calculated overall similarity and provide the user with a summary report.

13. The computer system of claim 8, further comprising:
determining that a lower edit distance value indicates a greater degree of similarity between the first trial and the second trial.

14. The computer system of claim 8, wherein immediate parents or children of the extracted concepts are searched together when the corpus of the existing trial is searched.

15. A computer program product for measuring the similarity of clinical trials, the computer program product comprising:
one or more computer-readable tangible storage media and program instructions stored on at least one of the one or more tangible storage media, the program instructions executable by a processor of a computer to perform a method, the method comprising:
receiving user-entered clinical trial data;
extracting concepts and values from each section of the received clinical trial data using natural language processing;
searching a corpus of existing trials with concepts similar to the extracted concepts and values;
computing an edit distance for each section of the existing trial;
calculating an overall similarity score based on a weighted distance of each section of the existing trial; and
displaying the overall similarity score to a user on a graphical user interface.

16. The computer program product of claim 15, further comprising:
initializing arrays, wherein arrays consist of a first array for a concept of a first trial, a second array for the second concept of a second trial, and a shared concept array for shared concepts between the first and second trial;
removing items from the first array for the concept of the first trial and the second array for the second concept of the second trial;
calculating an edit cost for each removed item; and
accumulating the calculated edit cost.

17. The computer program product of claim 15, further comprising:
using ontology to find related concepts between a first array for the concept of the first trial and the second array for the second concept of the second trial;
removing the shared concepts from the first array for the concept of the first trial and the second array for the second concept of the second trial;
calculating an edit cost for each removed item; and
accumulating the calculated edit cost.

18. The computer program product of claim 15, further comprising:
comparing concepts remaining in a first array for the concept of the first trial and the second array for the second concept of the second trial, wherein the remaining concepts are concepts other than the shared concepts and matched concepts;
removing the remaining concepts from the first array for the concept of the first trial and the second array for the second concept of the second trial;
calculating an edit cost for each removed item; and
accumulating the calculated edit cost.

19. The computer program product of claim 15, further comprising:
generating a summary of the calculated overall similarity and provide the user with a summary report.

20. The computer program product of claim 15, wherein immediate parents or children of the extracted concepts are searched together when the corpus of the existing trial is searched.
